(19) United States
(12) Patent Application Publication
(10) Pub. No.: US 2008/0193985 A1
(43) Pub. Date: Aug. 14, 2008

(54) Method For Recovering a Basic Amino Acid Form a Fermentation Liquor ll

(75) Inventors: Jong-Soo Choi, Seoul (KR); Tae-Hui Kim, Jeonbuk (KR); Sung Hyun Kim, Jeonbuk (KR)

Correspondence Address: CONNOLLY BOVE LODGE & HUTZ, LLP, P O BOX 2207, WILMINGTON, DE, 19899, US

(73) Assignee: BASF Aktiengesellschaft Patents, Trademarks and Licenses, Ludwigshafen (DE)

(21) Appl. No.: 11/918,502

(22) PCT Filed: Apr. 13, 2006
(86) PCT No.: PCT/EP2006/003430

§ 371 Date: Nov. 5, 2007

(30) Foreign Application Priority Data
Apr. 15, 2005 (DE) 10 2005 017 508.2

(51) Int. Cl. C12P 13/08; C12P 13/04

(52) U.S. Cl. 435/115; 435/106

(57) ABSTRACT

The invention relates to a method for recovering a basic amino acid from the fermentation liquor of a micro-organism strain that produces the basic amino acid. According to said method: a) the fermentation liquor is acidified with an acid, whose pKs value in water at 25° C. ranges between 2 and 5; and b) the basic amino acid is separated from the aqueous liquor obtained in step a) by the successive charging of a single or multiple stage, serial arrangement of a strongly acidic cation exchanger in the form of a salt with the liquor that has been obtained in step a) and the subsequent elution of the basic amino acid using a basic eluant.

The present invention relates to a method for producing a basic amino acid from the fermentation broth of a microorganism strain producing the basic amino acid.

Basic amino acids such as L-lysine, L-histidine, L-arginine and L-ornithine are predominantly produced by microbial fermentation methods (see e.g. Axel Kleemann et al., “Amino acids”, in “Ullmann's Encyclopedia of Industrial Chemistry”, 5th Edition on CD-ROM, 1997 Wiley-VCH and literature cited there; Th. Hermann, J. Biotechnol. 104 (2003), pp. 155-172 and literature cited there; Pfefferle et al., Adv. Biochem. Eng./Biotechnology, Vol. 79 (2003), 59-112 and literature cited there, and also Atkinson et al., in Biochemical Engineering and Biotechnology Handbook, 2nd ed., Stockton Press, 1991, Chapter 20 and literature cited there).

In the case of such fermentation methods, primarily an aqueous fermentation broth is obtained which, in addition to the desired basic amino acid and the biomass resulting from the microorganisms used, comprises a multiplicity of byproducts and impurities, e.g. other amino acids, substrate residues, salts, products of cell lysis and other byproducts.

The production of basic amino acids from the fermentation broth, and its purification are frequently performed using strongly acidic cation exchangers (see e.g. Th. Hermann, loc. cit; Atkinson et al, loc. cit.). For this purpose, the aqueous fermentation broth, before or after removing the microorganisms and other insoluble constituents (biomass), is acidified with a strong acid, for example sulfuric acid, to a pH below 2, so that the basic amino acid is present as dication. The acidified aqueous broth is then passed through a strongly acidic cation exchanger, the acid groups of which are present in the salt form, e.g. as sodium or ammonium salts, as a result of which the dication of the basic amino acid is adsorbed to the ion-exchange resin. The cation exchanger loaded with the basic amino acid is usually washed thereafter with water to remove impurities. The basic amino acid is then eluted by treatment with a dilute aqueous base, for example sodium hydroxide solution, ammonia water or an aqueous ammonium buffer, the salt form of the cation exchanger being regenerated at the same time. From the eluate thus produced, the basic amino acid, if appropriate after acidifying the eluate, is isolated in a conventional manner, e.g. by crystallization.

Of course, the liquid (effluent) flowing off when the cation exchanger is being loaded with the dication of the basic amino acid has a high salt concentration and is therefore also frequently termed high density waste water (HDWW). Also, the, if appropriate, succeeding wash step produces large amounts of water having a salt loading (low density waste water (LDWW)). These waste waters, to decrease the salt loading, must be subjected to complex waste water treatment. Alternatively, the salty waste waters can be dewatered and the resultant concentrate can be disposed of or fed to another use. However, both measures are associated with additional expenditure in terms of apparatus and high energy consumption and therefore contribute to a not inconsiderable extent to the costs of the fermentative amino acid production. There has therefore been no lack of attempts to reduce the salt loading and the amount of waste water which are produced in a workup by cation exchangers of fermentation broths comprising basic amino acid.

A further disadvantage is the precipitate formation occurring on acidification, which precipitate formation can lead to blockage of the cation-exchange arrangement. Moreover, additional wash steps are required to remove the impurities from the cation-exchange materials.

U.S. Pat. No. 4,714,767 describes a multistage method for separating off basic amino acids from an aqueous broth by means of an arrangement of a plurality of series-connected cation-exchange columns in which the last part of the effluent produced on loading the first column is recirculated to the loading operation of a later separation. It is also proposed that the last part of the eluate of the first column is recirculated to the elution process of a later separation. In this manner the amount of water is reduced, but not the salt loading.

Hsiao et al., Biotechnology and Bioengineering Vol. 49 (1996) pp. 341-347 propose, to reduce the amount of waste water, recirculating, to the fermentation medium, the salty effluents produced at the cation exchanger. In addition to the risk that as a result fermentation inhibitors which are customarily formed as byproducts in the metabolism of the microorganisms accumulate in the fermentation medium, it has been found that in this case the binding capacity of the cation exchanger is decreased so that the cost savings achieved by the reduced amount of waste water are consumed by the costs of a larger cation exchange arrangement.

I. Lee et al., Enzyme and Microbiol. Technol. 30 (2002) pp. 798-803, to reduce the salt loading in the workup of lysine-containing fermentation broths, propose the use of ion-exclusion chromatography instead of the cation exchanger customarily used. For this, first the solids content of the fermentation broth is removed by means of microfiltration. The resultant aqueous lysine-containing broth is adjusted to the isoelectric point (pH 9.74) and then passed through a cation exchanger. Since the ionic constituents of the broth are not absorbed, these are recovered in the effluent. The amino acid is then eluted with water. However, it has been found that a high recovery rate of L-lysine of greater than 90% is only achieved when not only is the rate of lysine-containing feed low, but also the through-flow rate. Despite the lower salt loading, therefore, this embodiment is not economic.

The object therefore underlying the present invention is to provide a method for producing basic amino acids from the fermentation broth of a microorganism strain producing the basic amino acid, which method overcomes the disadvantages described of the prior art and which in particular permits the reduction of the wash water and amount of salt produced and at the same time can be carried out with high efficiency, i.e. permits a high recovery rate of basic amino acid even at high loading and flow rates.

It has surprisingly been found that this object is achieved by a method in which
a) the fermentation broth is acidified using an acid, the pKₐ of which in water at 25° C. is in the range from 2 to 5, and
b) the basic amino acid from the aqueous broth obtained in step a) is separated off by successive loading of a single-stage or multistage serial arrangement of a strongly acidic cation exchanger in its salt form with the broth obtained in step a) and eluting the basic amino acid with a basic eluent.

Accordingly, the present invention relates to the method presented here and in the claims for producing a basic amino acid from the fermentation broth of a microorganism strain producing the basic amino acid.

The inventive method is associated with a number of advantages: firstly, on account of the acid selected in step a), there is no occurrence of significant precipitation of impurities which can block the cation exchanger and thus increase the wash water requirement. In addition, the amount of salt produced in the inventive method, and thus the salt loading of the waste water, are lower than in the methods of the prior art in which cation exchangers are used to separate off and produce the basic amino acid from the fermentation broth. In addition, high yields of generally greater than 95% of basic amino acid are achieved even at high loadings and through-flow rates at the cation exchanger.

According to the invention, in a first step, the fermentation broth is acidified with an acid, the pKa of which is, at 25° C., in the range from 2 to 5, and in particular in the range from 3 to 4. It goes without saying that the acid used is inert, that is to say does not cause a chemical change in the amino acid to be isolated, apart from a protonation.

Examples of suitable acids comprise phosphoric acid, organic monocarboxylic acids preferably having from 1 to 6 carbon atoms, such as formic acid, acetic acid, propionic acid, butyric acid and pentanoic acid, dicarboxylic acids preferably having from 2 to 6 carbon atoms, such as oxalic acid, malonic acid, maleic acid, fumaric acid, itaconic acid, succinic acid, glutaric acid, adipic acid and sorbic acid, hydroxycarboxylic acids preferably having from 1 to 3 carboxyl groups, and also having at least one, e.g. 1, 2, 3 or 4, hydroxyl groups, such as citric acid, glycolic acid and lactic acid, and mixtures of these acids. Preferably, the acid is selected from the group consisting of the organic carboxylic acids and hydroxycarboxylic acids. In a particularly preferred embodiment of the invention, the acid is formic acid.

The amount of acid is preferably selected in such a manner that a pH of from 3.5 to 6.0, and in particular in the range from pH 4.0 to 5.5, results in the broth. Preferably, for the acidification, use is made of from 0.05 to 2 mol of acid, in particular from 0.1 to 1 mol of acid, and especially from 0.15 to 0.5 mol of acid, per kg of fermentation broth.

If appropriate, before or after the acidification, a part or the main amount of the microorganisms and if appropriate other solids present in the fermentation broth can be separated off from the fermentation broth. Separation of these constituents is not necessary in principle. Therefore, in a preferred embodiment of the invention, these constituents are not separated off and the cation-exchange arrangement is loaded directly with the acidified aqueous broth.

The separation of the microorganisms and other solid constituents can, if desired, be performed in customarily for the separation of microorganisms by filtration including cake- and depth-filtration, cross-flow filtration, by membrane separation methods such as ultra- and microfiltration, by centrifugation and decanting, by using hydrocyclones, or in another manner. Before the separation, it has proved to be useful to inactivate the microorganisms in the fermentation broth (sterilizing the fermentation broth), for example by customary pasteurization methods such as by introducing heat and/or hot steam. For this, conventional heat exchangers, for example shell-and-tube heat exchangers or plate heat exchangers can be used.

From the aqueous broth obtained in step a), then, in step b), the basic amino acid is separated off using a cation exchange arrangement. The separation in step b) comprises according to the invention at least one loading step, in which the basic amino acid is adsorbed to the strongly acidic ion exchanger, and at least one elution step, by which the basic amino acid is desorbed from the ion exchanger. These steps can be repeated several times in the stated sequence and, between the steps, wash steps with water can be carried out.

The cation exchange arrangement used in the inventive method comprises one or more, e.g. 2, 3 or 4, series-connected stages, customarily in the form of ion exchange columns which, as stationary phase, comprise one or more strongly acid cation exchangers.

As strongly acidic cation exchangers, in principle all ion-exchange resins come into consideration which have strongly acidic groups, generally sulfonate groups. Generally, these are particulate, moderately or greatly crosslinked organic polymers, frequently based on polystyrene, which have on the surface of the polymer particles a multiplicity of strongly acidic groups. The mean number of acidic groups is customarily in the range from 1 to 4 meq/ml of ion-exchange resin. The mean particle size of the ion-exchange particles is typically in the range from 0.1 to 1 mm, with larger and also smaller particle sizes also being able to be suitable depending on the dimensioning of the ion-exchange arrangement. The polymer particles can be, e.g., gel-like or have a macroporous structure.

Such ion exchangers are known and some are offered commercially for purifying amino acids, for example under the tradenames Lewatit® K or Lewatit® S from Bayer Aktiengesellschaft, e.g. Lewatit® K 2629, Lewatit® S110, Lewatit® S110H, Lewatit® S1467, Lewatit® S1468, Lewatit® S2568, Lewatit® S2568H, Amberjet®, Amberlyst® or Amberlite® from Rohm & Haas, e.g. Amberjet® 1200, Amberjet® 1500, Amberlite® 200, Amberlite® 250, Amberlite® IRV120, Amberlite® IR 120, Amberlite® IR 200C, Amberlite® CG 6000, Amberlyst® 119 Wet, Dowex® from Dow Chemicals, e.g. Dowex® 50X1-100, Dowex® 50X2-100, Dowex® 50X2-200, Dowex® 50X2-400, Dowex® 50X4-100, Dowex® 50X4-200, Dowex® 50X4-400, Dowex® 50X8-100, Dowex® 50X8-200, Dowex® 50X8-400, Dowex® 40X1-100, Dowex® 40X1-100, Dowex® 40X1-100, Dowex® HCR-S, Dowex® HCR-W2, Dowex® MSC-1, Dowex® 650C, Dowex® G26, Dowex® 88, Dowex® Monosphere 88, Dowex® Monosphere 99K/320, Dowex® Monosphere 99K/350, Dowex® Monosphere 99Ca/320, Dowex® Marathon C, Dowex® 032, Dowex® 406, Dowex® 437, Dowex® C500ES, Dowex® XUS 43518, Dowex® XUS 40406.00, Diaion® from Mitsubishi Corp., e.g. Diaion® SK1B, Diaion® SK1BS, Diaion® SK104, Diaion® SK112, Diaion® SK116, Diaion® 1-3561, Diaion® 1-3565, Diaion® 1-3570, Diaion® 1-3573, Diaion® 1-3577, Diaion® 1-3581, Duolite® D 5427, Duolite® D 5552 (organically based cation exchanger), and in addition Adsorbosphere® SCX, Bakerbond® SCX, Partisil® SCX, Spherisorb® SCX, Supelcosil® LC3-SCX, Ultralsil® SCX and Zorbax® 300 SCX (silica-based cation exchanger).

The cation-exchange arrangement can be operated batchwise and then has one or more, e.g. 2, 3 or 4, series-connected stationary ion-exchange fixed beds. It can also be operated continuously and then has generally 5 to 50, and in particular 15 to 40, ion-exchange beds, which can be, e.g., constituent of a “true moving bed” arrangement (see K. Tekeuchi J. Chem. Eng. Japan 11 (1978 pp. 216-220), a “continuous circulating annular” arrangement (see J. P. Martin, Discuss. Farraday Soc. 1949, p. 7) or of a “simulated moving bed” arrangement, as described in, for example, U.S. Pat. No. 2,985,589, WO 01/72689 and also by G. J. Rossiter et al. Proceedings of AIChE Conference, Los Angeles, Calif., November 1991, or H. J. Van Walsem et al. J. Biochtechnol. 59 (1997) pp. 127-123.

Before the loading of the cation exchanger with the basic amino acid which is to be separated off, the cation exchange material contained in the ion-exchange arrangement is in its salt form, i.e. the strongly acidic groups of the cation exchanger are in deprotonated form and coordinate to give charge neutrality to a corresponding number of cations. Generally, the cations are alkali metal cations, in particular sodium ions or, particularly preferably, ammonium ions (NH₄⁺).

In order to load the cation exchanger with the basic amino acid, the acidified aqueous broth is passed through the cation-exchange arrangement in the usual manner. The loading can be performed not only in a descending manner but also ascending manner, with the former being preferred. The loading is preferably performed at a specific flow rate in the range from 0.1 h⁻¹ to 2 h⁻¹. The loading is preferably performed at a temperature in the range from 20 to 70° C., and in particular in the range from 30 to 60° C. The amount of aqueous broth is customarily selected so that at least 35%, and in particular at least 42%, of the basic amino acid present in the aqueous broth is adsorbed. The amount of aqueous broth is generally 0.8 to 2 times the amount of the bed volume. Depending on the degree of adsorption, the effluent produced at the exit of the cation-exchange arrangement can still comprise basic amino acid, so that the effluent, if appropriate after adjusting the pH, can be passed to an ion exchanger in a subsequent stage.

The loading process can be followed by a wash step. For this, water is passed through the cation-exchange arrangement. The amount of wash water is, at this stage, customarily 0.05 to 0.3 times the bed volume. The resultant wash waters can comprise small amounts of the basic amino acid and can then be combined with the effluent produced on loading. In contrast to the methods of the prior art, such a wash step is not necessary, so that a preferred embodiment of the inventive method does not comprise a wash step and the elution is performed directly after loading.

The loading step, or the wash step carried out if appropriate, is followed by the elution of the basic amino acid. For this, an aqueous solution of a base (eluant) is passed through the cation-exchange arrangement. As a result the basic amino acid is desorbed and is eluted, and the cation exchanger is regenerated, i.e. the acidic groups of the cation exchanger are converted back to the salt form. The base concentration in the eluant is customarily in the range from 1 to 10% by weight, and in particular in the range from 2 to 8% by weight. Suitable bases are, for example, ammonia, alkali metal hydroxides and alkali metal carbonates, with sodium hydroxide solution and, in particular, ammonia being preferred. The amount of aqueous base is generally 0.5 to 3 times the amount of the bed volume. With regard to the temperatures and flow rate, that said for loading applies. The elution can be carried out not only in the ascending but also descending manner. The elution can be carried out in the same direction as loading or in the opposite direction to this.

The elution can be followed by a further wash step, if appropriate to remove impurities present. For this, water is passed through the cation-exchange arrangement. The amount of wash water at this stage is customarily 0.05 to 0.3 times the bed volume. The effluent produced in the wash step is fed as waste water of low salt loading to a customary waste water treatment, or to another workup.

The eluate produced in the elution is worked up in a customary manner to produce the amino acid. Generally, for this, the eluate will be concentrated, e.g. by removing the water in a customary evaporator arrangement.

In this manner a concentrated aqueous solution of the basic amino acid is obtained, from which the basic amino acid can be isolated as hydrochloride, e.g., after addition of hydrochloric acid, by precipitation or crystallization. Methods for this are known to those skilled in the art and are extensively described in the literature (e.g. Hermann, T. Industrial Production of amino acids by coryneform bacteria, J. of Biotechnology, 104(2003), 155-172).

The aqueous condensate produced in the concentration can be discarded or recirculated to the process. For example, the condensate can be recirculated to the elution step of the basic amino acid in a subsequent amino acid separation. Preferably, for this, the condensate, after the elution with the aqueous base, is passed through the cation-exchange arrangement. The resultant effluent frequently still comprises small amounts of basic amino acid and is customarily recirculated to the elution of a subsequent amino acid separation.

The inventive method is applicable in principle to the isolation of all basic amino acids, in particular natural amino acids such as lysine, ornithine, histidine or arginine and is used, in particular, for isolating L-lysine produced by fermentation.

The type of the fermentation process and also of the microorganism strain used for producing the amino acid play no role for the inventive method, so that the inventive method is suitable for isolating the basic amino acid from any desired fermentation broths. Generally, such methods are involved in which a microorganism strain which produces the desired basic amino acid is cultured in a fermentation medium which comprises, as substrate, at least one carbon source, e.g. molasses and/or raw sugar, and a nitrogen source, e.g. ammonia or ammonium salts such as ammonium sulfate, and also if appropriate minerals and trace elements. These substrate constituents can be used as such or in the form of a complex mixture, e.g. as corn-steep liquor.

The type of microorganism strain obviously depends on the type of amino acid to be produced. Generally, these are strains which overproduce the desired basic amino acid. In the case of L-lysine, ornithine and histidine, these are generally strains of the genus Corynebacterium or Brevibacterium, e.g. of the species Corynebacterium glutamicum or Brevibacterium lactofermentum, in the case of arginine, strains of the species Bacillus subtilis or Brevibacterium flavum, with, however, recently strains of other species being used.

Generally, the fermentation is carried out until the content of basic amino acid in the fermentation broth is in the range from 50 to 200 g/l, and in particular in the range from 80 to 150 g/l. The content of biomass, i.e. microorganisms (as biodrymass) and other insoluble constituents of biological origin (e.g. cellulose fibers from the glucose source), is customarily in the range from 3 to 7% by weight. In addition, the fermentation broth generally further comprises residual amounts of substrate, e.g. unconsumed sugars (usually less than 40 g/l), and also byproducts of fermentation, e.g. acidic or neutral amino acids or other basic amino acids, peptides and the like. The pH is frequently in the range from >6 to 7.5, and in particular in the range from 6.2 to 7.2. In addition, the fermentation broth generally further comprises residual amounts of substrate, e.g. unconsumed sugars (customarily less than 40 g/l) and also byproducts of the fermentation, e.g. acidic or neutral amino acids or other basic amino acids, peptides and the like.

The fermentation methods can be carried out continuously or batchwise as batch or fed-batch methods. Generally, the methods relate to a fermentation broth which was produced by a fed-batch method, i.e. the majority of the substrate is fed to the microorganism-containing broth in the course of the fermentation.

Such methods and suitable microorganism strains are known to those skilled in the art, e.g. from the prior art cited at the outset (see, in particular, Pfefferle et al. and Th. Herrmann, loc. cit), and also WO 95/16042, WO 96/06180, WO 96/16042, WO 96/41042, WO 01/09306, EP-A 175309, EP-A 327945, EP-A 551614, EP-A 837134, U.S. Pat. No. 4,346,170, U.S. Pat. No. 5,305,576, U.S. Pat. No. 6,025,165, U.S. Pat. No. 6,653,454, DE 253199, GB 851396, GB 849370 and GB 1118719 (production of L-lysine), EP-A 393708, GB 1098348, U.S. Pat. No. 3,668,072, U.S. Pat. No. 3,574,061, U.S. Pat. No. 3,532,600, U.S. Pat. No. 2,988,489, JP 2283290, JP 57016696 (L-ornithine), U.S. Pat. No. 3,902,967, U.S. Pat. No. 4,086,137, GB 2084566 (arginine) U.S. Pat. No. 3,875,001 and U.S. Pat. No. 3,902,966 (histidine).

The measures for carrying out and controlling such fermentations technically are familiar to those skilled in the art and can be found in the relevant literature, for example Storhas (see above) and J. E. Bailey et al. Biochemical Engineering Fundamentals, 2nd ed. MacGraw-Hill 1986, Chapter 9.

The invention will be described by the following examples and comparative examples which show preferred embodiments of the inventive method and are not to be understood as restricting.

Abbreviations Used:
HDWW: high density waste water
LDWW: low density waste water
ID: inner diameter
H: height
Lys-HCl: L-lysine monohydrochloride
BV: bed volume (volume of the cation exchanger in the arrangement)
SV: specific flow rate (flow velocity in 1 BV/H)

Materials Used:

All experiments were carried out using an L-lysine-containing fermentation broth which was produced in a manner known per se by fermentation with C. glutamicum. The fermentation broth had a content of Lys-HCl of 110 to 130 g/l and a content of biomass (calculated as biodrymatter) of 2.5-3.5% by weight. The salt content was between 3 and 5% by weight.

Cation-Exchange Arrangement

In comparative example 1 and examples 1 and 2, as cation-exchange arrangement served in each case a cylindrical column of dimensions 125 mm (ID)×495 mm (H) which was loaded with 3000 ml of a strongly acidic cation-exchange resin. As cation exchanger, use was made of a sulfonated crosslinked polystyrene of the gel type having a mean particle size of about 0.6 mm (DIAION SK1B from Samyang Co. Ltd. Korea) and a total capacity >2 meq/ml. The cation-exchange arrangement was equilibrated before its use with 6% strength by weight aqueous ammonia.

In example 3 there served as cation-exchange arrangement a SepTor pilot plant (Model 30-6, Torus Liquid Separation, Holland), which was loaded with 22.8 l of strongly acidic cation-exchange resin. As cation exchanger there served a sulfonated crosslinked polystyrene of the gel type having a mean particle size of about 0.6 mm (DIAION SK1B from Samyang Co. Ltd. Korea) and a total capacity >2 meq/ml.

### COMPARATIVE EXAMPLE 1

A fermentation broth having a lysine content of 12.5% by weight and a biomass content of 3% by weight was acidified to a pH of 1.5 using 5.8 g of concentrated sulfuric acid per 100 g of broth. 5.5 l of the acidified fermentation broth were passed from bottom to top through the cation-exchange arrangement at a temperature of 45° C. at a specific flow rate of 1 h⁻¹. The amount of lysine present in the broth passed through corresponded to 210 g of Lys-HCl per liter of ion-exchange resin. Thereafter, the column was rinsed with 3.4 l of water at a specific flow rate of 2 h⁻¹. The column was then allowed to run empty. The resultant effluent was collected and the amount of lysine herein determined. From this an amount of adsorbed lysine of 95.7 g per liter of resin was calculated.

Then, for the elution of the L-lysine, 6 l of a 3 w/v % strength aqueous ammonia solution was passed from top to bottom through the cation-exchange arrangement at a specific flow rate of 1 h⁻¹.

The lysine content of the eluate was determined. From this a recovery rate of 98% was calculated, based on adsorbed Lys-HCl.

### EXAMPLE 1

A fermentation broth having a lysine content of 12.5% by weight and a biomass fraction of 3% by weight was acidified to a pH of 4.1 using 1 g of 87% strength by weight formic acid per 100 g of broth. 5.5 l of the acidified fermentation broth were passed from top to bottom through the cation-exchange arrangement at a temperature of 45° C. at a specific flow rate of 1 h⁻¹. The amount of lysine in the broth passed through corresponded to 210 g of Lys-HCl per liter of ion-exchange resin. The resultant effluent was collected and the amount of lysine herein determined. From this an amount of adsorbed lysine was calculated as 88.2 g per liter of resin.

Then, for the elution of the L-lysine, 6 l of a 3 w/v % strength aqueous ammonia solution were passed through the cation-exchange arrangement from top to bottom at a specific flow rate of 1 h⁻¹. The column was rinsed with 0.7 l of water at a specific flow rate of 1 h⁻¹.

The eluate was collected and the lysine content determined. From this a recovery rate of 98% was calculated, based on adsorbed Lys-HCl. A wash step was not required.

### EXAMPLE 2

A fermentation broth having a lysine content of 12.5% by weight and a biomass fraction of 3% by weight was acidified to a pH of 3.6 using 3 g of 87% strength by weight formic acid per 100 g of broth and the cell mass was separated off by centrifugation. The resultant broth comprised <0.5% by weight of cells. 4 l of this aqueous broth were passed through the cation-exchange arrangement from top to bottom at a temperature of 45° C. at a specific flow rate of 1 h⁻¹. The amount of lysine in the broth passed through corresponded to 170 g of Lys-HCl per l of ion-exchange resin. The resultant effluent was collected and the amount of lysine determined. From this an amount of adsorbed lysine was calculated as 107 g per liter of resin.

Then, for the elution of the L-lysine, 6 l of a 6 w/v % strength aqueous ammonia solution were passed from top to bottom through the cation-exchange arrangement at a specific flow rate of 1 h⁻¹ at a temperature of 45° C.

The lysine content of the eluate was determined. From this a recovery rate of 95% was calculated, based on adsorbed Lys-HCl.

### EXAMPLE 3

A fermentation broth having a lysine content of 12.5% by weight and a biomass fraction of 3% by weight was acidified to a pH of 3.2 using 6 g of 87% strength by weight formic acid per 100 g of broth and the cell mass was separated off by centrifugation. The resultant broth comprises <0.5% by weight of cells. 19.1 l of this aqueous broth were passed from top to bottom through the cation-exchange arrangement at a temperature of 45° C. at a specific flow rate of 0.36 h⁻¹. The amount of lysine in the broth passed through corresponded to 105 g of Lys-HCl per l of ion-exchange resin. The resultant effluent was collected and the amount of lysine determined. From this an amount of adsorbed lysine of 100 g per liter of resin was calculated.

Then, for the elution of the L-lysine, 45.6 l of a 6 w/v % strength aqueous ammonia solution were passed from top to bottom through the cation-exchange arrangement at a temperature of 45° C. at a specific flow rate of 0.36 h⁻¹.

The lysine content of the eluate was determined. From this a recovery rate of 95% was calculated, based on adsorbed Lys-HCl.

1. (canceled)

2. (canceled)

3. (canceled)

4. (canceled)

5. (canceled)

6. (canceled)

7. (canceled)

8. (canceled)

9. (canceled)

10. A method for producing a basic amino acid from the fermentation broth of a microorganism strain producing the basic amino acid, the method comprising:
a) acidifying the fermentation broth using an acid, the pKa of which in water at 25° C. is in the range from 2 to 5, and
b) separating the basic amino acid from the aqueous broth obtained in step a) by loading the broth obtained in step a) onto a single-stage or multistage serial arrangement of a strongly acidic cation exchanger in its salt form, and eluting the basic amino acid with a basic eluent;
wherein microorganisms and other solid constituents present in the fermentation broth are not separated before the loading of the single-stage or multistage serial arrangement of the strongly acidic cation exchanger.

11. The method according to claim 10, wherein the acid is selected from the group consisting of organic carboxylic acids and hydroxycarboxylic acids.

12. The method according to claim 11, wherein the acid is formic acid.

13. The method according to claim 10, wherein from about 0.05 to 2 mol of acid per kg of fermentation broth is used for the acidification.

14. The method according to claim 10, wherein the fermentation broth is acidified to a pH from about 3.5 to 6.0.

15. The method according to claim 10, wherein the acid groups of the ion exchanger before the loading are present in a form selected from the group consisting of sodium and ammonium salt groups.

16. The method according to claim 10, wherein the basic amino acid is eluted with aqueous ammonia or sodium hydroxide solution or a mixture thereof.

17. The method according to claim 10, wherein the basic amino acid is lysine.

18. The method according to claim 10, additionally comprising the isolation of the basic amino acid by crystallization from the eluate.
